(19) United States
(12) Patent Application Publication
(10) Pub. No.: US 2004/0133092 A1
(43) Pub. Date: Jul. 8, 2004

(54) Wireless system for measuring distension in flexible tubes

(76) Inventor: Aron Z. Kain, Wesley Hills, NJ (US)

Correspondence Address: KATTEN MUCHIN ZAVIS ROSENMAN, 575 MADISON AVENUE, NEW YORK, NY, 10022-2585, US

(21) Appl. No.: 10/470,409

(22) PCT Filed: Mar. 27, 2002
(86) PCT No.: PCT/US02/09543

(51) Int. Cl. A61B 5/04; A61B 8/06

(52) U.S. Cl. 600/377; 600/453; 128/903

(57) ABSTRACT

A wireless sensing system is configured to measure the distension of a flexible tube. The system includes a sensing unit having a sensor element that attaches to the tube and which causes a quiescent frequency produced by the sensing unit to change when the sensor element is physically distorted by distension of the tube. A scanning unit of the system remotely and wirelessly triggers the sensing unit to power up and transmit a modulated signal to the scanning unit for decoding, where the decoded signal indicates a measured quiescent frequency of the sensing unit. The wireless sensing system may be employed, for example, to measure the distension of a blood vessel for the purpose of monitoring blood pressure.

### CROSS REFERENCE TO RELATED APPLICATIONS

The present application claims priority under 35 U.S.C. § 119(e) from U.S. Ser. No. 60/279,018, filed on Mar. 27, 2001, and U.S. Ser. No. xx/xxx,xxx, filed on Feb. 20, 2002. Both priority applications were filed by an inventor common to the present application, and are hereby incorporated by reference.

### FIELD OF THE INVENTION

This invention relates generally to remote measurement devices, and more particularly to an implantable system for measuring distension in a flexible tube.

### BACKGROUND OF THE INVENTION

The methodology and technology used to measure blood parameters in living beings is well known in the arts, and can be characterized by either invasive or noninvasive techniques. Such techniques are well summarized by U.S. Pat. No. 6,015,386 to Kensey et al., which is incorporated herein by reference. Conventional noninvasive systems for measuring blood pressure of a living being typically require a pressurized occlusive cuff and means to monitor and analyze the resulting Korotkoff sound or the oscillometric pressure variations. These systems typically exhibit inaccuracies and drift due to local tissue accommodation and/or possible nonlinear and viscoelastic effects of the blood vessel tissue.

Invasive systems require direct access to the interior of the blood vessel via an arterial puncture. Such systems are usually not feasible for a series of long-term measurements, as hospital stays and close monitoring by skilled medical staff is most likely required. Moreover, transcutaneous or percutaneous access to the interior of the blood vessel to measure/monitor the pressure therein carries with it various health risks inherent in any arterial puncture, particularly where wires have to extend through the arterial wall and the intervening tissues for an extended period of time.

Other types of implantable devices for monitoring the blood pressure of a living being (e.g., human or animal) have been disclosed in the patent literature. See for example, U.S. Pat. No. 3,189,023 to Salz et al. The Salz patent requires that electrical conductors (wires) extend out through the body of the being from an implanted unit extending about the blood vessel, providing drawbacks from the standpoint of restriction of mobility, resistance to infection, and discomfort.

The Kensey patent discloses a method for monitoring blood pressure without requiring internal arterial access. The system comprises a sensor/transducer unit and an associated energy application/transceiver unit. The sensor/transducer unit is adapted to be implanted at the radial artery immediately proximally of the wrist. The energy application/transceiver unit is arranged to be located externally of the body of the being but adjacent to the site of the implanted sensor/ transducer unit to selectively provide energy to the sensor/transducer unit to activate that unit and to receive wireless signals representative of the being's blood pressure therefrom. The sensor/transducer unit includes a housing for surrounding at least a portion of the wall of the blood vessel when implanted. A portion of the housing serves to flatten a portion of the blood vessel's periphery. A deflection member, e.g., a probe having a ferrite core mounted on it, is located within the housing and is movable with respect thereto in response to pressure changes within the blood vessel. The deflection member is coupled to passive energy responsive means, (e.g., an inductor coil) so that movement of the core effects a change in the inductance of the coil located within the housing. The energy responsive means is arranged for providing an output signal, e.g., a wireless electromagnetic signal, representative of pressure changes within the blood vessel in response to energy applied thereto by the externally located energy applicator/transceiver unit. The energy applicator/transceiver unit is arranged to pick up or receive the wireless electromagnetic signal.

The Kensey device suffers from a number of shortcomings that limit the usefulness of the device to shallow arteries under ideal conditions. Since the implanted device is passive, energy coupling must be done over a very small distance in order for measurements to be done. This precludes the device from working in its taught form on deeper arterial and venous blood vessels such as the Aorta, Superior Vena Cava, or the Pulmonary arteries and veins. Also, the Kensey device is relatively large in volume and thereby requires a significant displacement of surrounding tissue in order to accommodate the implant. This is highly undesirable, for example, deep in the thoracic cavity. Furthermore, the device necessarily requires the forced distortion of the blood vessel, by as much as 35% in order to sense the internal arterial blood pressure. Although this might not be significant for the radial artery, as Kensey et al claim, it could be catastrophic and lethal for measuring the Aorta.

### SUMMARY OF THE INVENTION

These and other shortcomings in the prior art are overcome in a wireless system that allows the measurement of the distension of a flexible tube. The flexible tube can include biological materials such as blood vessels, as well as industrial materials such as PVC (polyvinyl chloride) or stainless steel. The construction, geometry, and topology of the tube are inconsequential to the workings of the system; the only requirement of the tube is that it distends in response to internal force or pressure. The cause of the force or pressure as well as the medium through which the force is transmitted to the walls of the tube (gas, liquid or solid) is also inconsequential to the system operation.

Applications of this system may for example range from the measurement of the internal blood pressure of an artery or vein sensed from either the outside or the inside of the blood vessel, to checking the ovality of steel pipes as they are buried in the ground for utility services, or to measuring the internal pressure in a pipe carrying Hydrocloric Acid from outside the pipe.

In a first embodiment of the present invention, a system is provided for monitoring the pressure within a blood vessel of a living being. The system comprises a sensor/transducer unit and an associated scanner/transceiver unit. The sensor/transducer unit is adapted to be implanted within the body of the being regardless of which blood vessel is chosen for monitoring. The blood vessel may be shallow or deeply located from the beings out skin layer. Multiple blood vessels can be monitored simultaneously as the system allows for direct identification of the individual blood vessel queried for information. The Scanner/transceiver unit is preferably arranged to be located externally of the body of the being.

The sensor/transducer unit includes a biocompatible, low profile, flexible housing for attachment to at least a portion of the wall of the blood vessel when implanted. The attachment can occur on the outside of the blood vessel as when a patient is undergoing open-heart surgery. Equally as applicable, the attachment can occur on the inside of the blood vessel as when a patient is undergoing catheterization, either to specifically implant this sensor, or as part of another catheterization procedure.

The housing and sensor are flexible enough so that no forced distension of the blood vessel is required in order for measurements to be made. The blood vessel is allowed to pulse and distend without being manipulated by the sensor/transducer. This is enabled by a sensor that is more compliant than the blood vessel. The flexible sensor distorts according to the internal blood pressure of the blood vessel. The sensor's distortion gives rise to a change in the quiescent frequency of the transducer. This in turn influences the frequency of the enclosed and proximal electronics. This change in frequency is directly correlated to the internal vessel blood pressure and is relayed, via wireless communication, to the external transceiver. The sensor/transducer's on-board energy source is either a self-contained battery or a mechanism that converts the mechanical motion of the blood vessel, organ, or living being into storable and useable electrical energy. Alternatively, the transducer may be combined with appropriate passive components such that the transducer is both frequency selective and re-radiates the RF energy that impinges on it so that a simplified fully passive embodiment of the frequency selectivity of the transducer with full telemetry capabilities is realized. This fully passive embodiment will be best realized when the transducer is to be used internal to the blood vessel, as would be the case in catheterization implantation.

### BRIEF DESCRIPTION OF THE DRAWINGS

A more complete understanding of the invention may be obtained by reading the following description of specific illustrative embodiments of the invention in conjunction with the appended drawing in which:

FIGS 1A, 1B illustrates a first embodiment of the inventive wireless measurement system;

FIGS. 2, 3 illustrate two embodiments of the present invention directed to measuring human blood pressure at the heart;

FIGS. 4A, 4B diagram elements of two embodiments of the sensor unit of the present invention;

FIG. 4C diagrams a telemetry signal output as embodied in FIG. 4A FIG. 5 illustrates several embodiments of a capacitive sensor element for the sensor unit of FIGS. 4A, 4B;

FIG. 5A illustrates fringe effects typical for the sensor elements of FIGS. 5 and 10;

FIG. 6 illustrates an exemplary attachment of a sensor element of FIG. 5 to a blood vessel;

FIG. 7 shows an alternative embodiment of the sensor unit of FIGS. 2, 3 providing a piezoelectric power source;

FIG. 8 provides a circuit diagram for the embodiment of FIG. 7;

FIG. 9 illustrates an alternative embodiment of the sensor unit of FIG. 1B employing a passive RF frequency power source;

FIG. 10 shows a variety of additional sensor topologies consistent with the principles of the present invention;

FIG. 11 presents a cross-sectional view of a hermetically sealed package containing an embodiment of the sensor unit of FIG. 1B;

FIGS. 12(a), 12(b) illustrate an exemplary attachment of a sensor element of FIG. 5 within a blood vessel;

FIGS. 13, 14 illustrate the operation of an embodiment of the present invention employing dual sensing elements;

FIG. 15 illustrates an additional embodiment of the present invention in which the sensor unit of FIG. 1B is encapsulated in a distendable, hermetically sealed capsule for positioning within a blood vessel.

### DETAILED DESCRIPTION OF THE PREFERRED EMBODIMENT

The following detailed description includes a description of the best mode or modes of the invention presently contemplated. Such description is not intended to be understood in a limiting sense, but to be an example of the invention presented solely for illustration thereof, and by reference to which in connection with the following description and the accompanying drawings one skilled in the art may be advised of the advantages and construction of the invention. While the embodiments disclosed are frequently explained with reference to the measurement of blood pressure in a biological entity, it should be understood that the present invention is broadly applicable to measuring a variety of effects that may be related to distension in a variety of types of members, including flexible tubes that are not necessarily biological in nature.

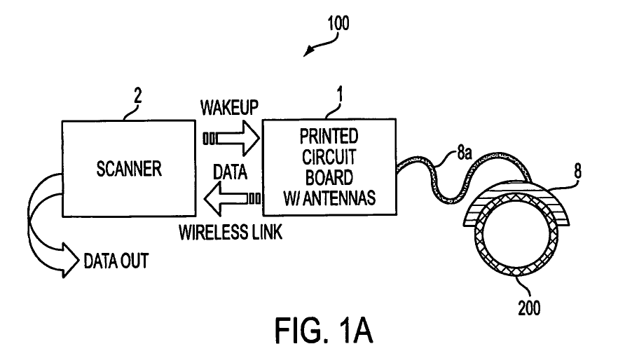

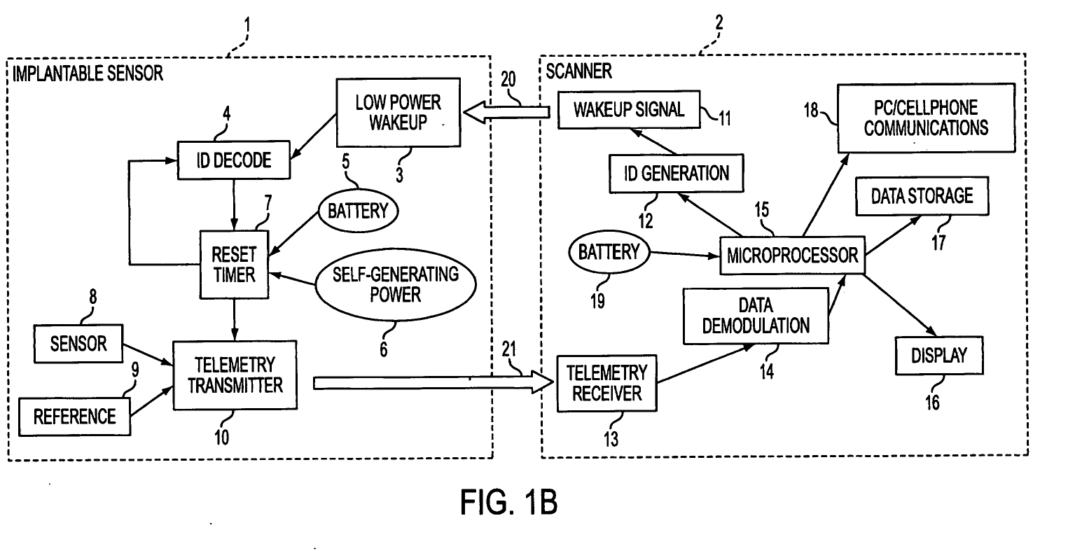

FIG. 1A indicates the principal elements that comprise the wireless measurement system 100. A sensor element 8 is embodied in a planar structure that is made flexible to conform to the shape of the tube 200. The sensing technology employed can be one of a number of planar technologies (such as described in U.S. Pat. No. 5,261,278, U.S. Pat. No. 5,546,806, U.S. Pat. No. 5,578,969, all issued to Kain), or even a conventional sensor technology (for example, piezoresitive), that is embedded in a flexible membrane that conforms to the outside of the tube 200. The sensor element 8 can be held in place by any means necessary (glue, tape, weld, etc.). The principal requirement for the sensor/element 8 membrane is that a measurable signal (for example, microwave resonance frequency in the case of the sensors disclosed by the patents of Kain, voltage in the case of piezo-resistive technology, etc) is produced for an arbitrary distension of the tube 200.

The measured (sensed) signal is then transmitted to electronics housed in sensor unit 1. The electronics can be remotely located and connected via a lead 8a (as shown) or co-located, for example, so that sensor element 8 and sensor electronics unit 1 are integrated together to form an application specific integrated circuit (ASIC). The electronics then process the sensed signal and convert it to a radio frequency (RF) signal that can be transmitted to a scanner 2.

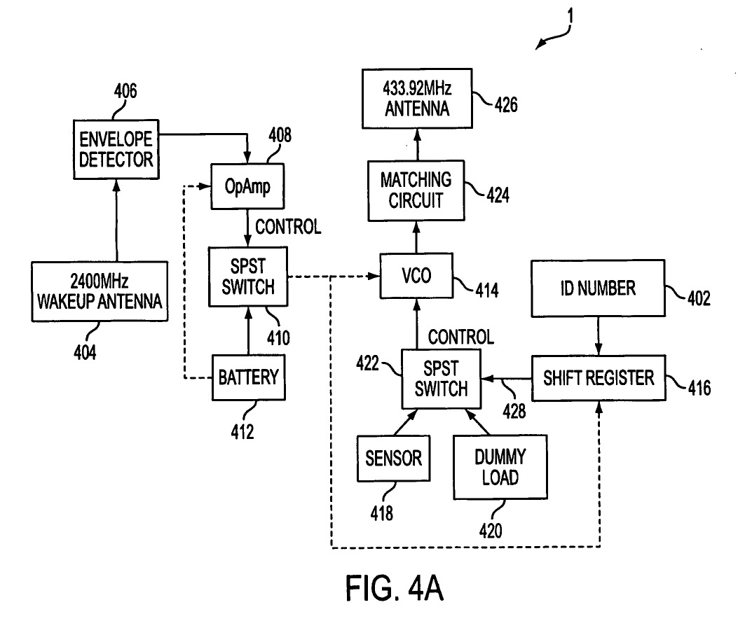

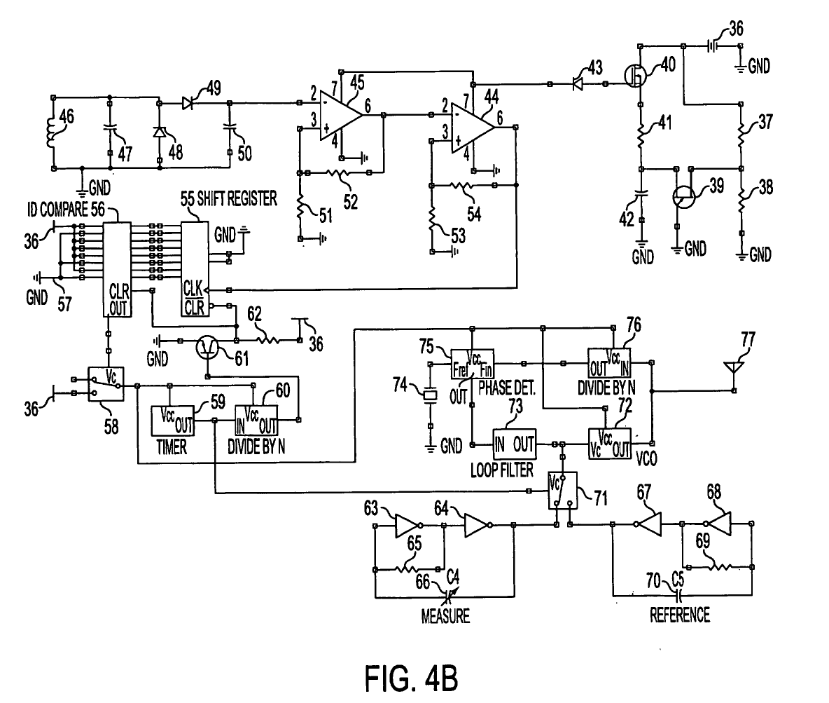

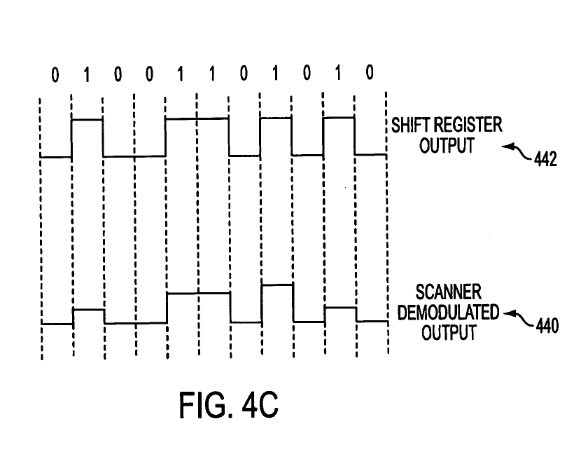

The scanner 2 ultimately reads the RF signal and decodes the required data. The scanner 2 can also initiate a wakeup of the sensor in a timed fashion in order, for example, to preserve battery lifetime in sensor unit 1. In self-powered systems that require no external energy storage such as a battery (for example, solar cell or mechanical motion energy conversion) a wakeup capability may not be included. The self-powered system might be continuously providing the required data or, equally well, be periodically woken to transmit the required data as taught herein. Furthermore, electronics unit 1 can consist entirely of passive and active components that simply transduce incoming RF energy from scanner 2 and convert it to useful energy for the sensor 8 to function properly, with out the need for any self-generating power capability. FIG. 1B shows a system diagram for the sensor unit 1 and the scanner 2 of FIG. 1A. A detailed circuit diagram of the implantable sensor 1 illustrated in FIG. 1B is shown in FIG. 4B. The scanner 2 provides a wakeup signal 20 that turns on the implantable sensor, as well as receives the data transmitted from the sensor, demodulates and processes the data for useful display, storage, or forwarding to other equipment that desires such data. The sensor unit 1 remains in a very low current consuming standby mode until woken up by the scanner 2 via wakeup signal 20. The sensor unit 1 measures and transmits measurement signals 21 for a predetermined amount of time and then returns to its “sleep” state.

Low power wakeup circuit 3 is a low current, low duty cycle oscillator that periodically turns on a circuit that looks for a wakeup signal 20 from the scanner 2. The received signal from the scanner is processed by an ID decoder 4 that validates the signal 20 to determine whether sensor unit 1 is the correctly addressed device. The decoder 4 then provides a signal that allows reset timer 7 to function. Reset timer 7 allows for both DC power distribution between the battery 5 or self generating power source 6 and the rest of the current consuming circuitry of the sensor unit 1, as well as providing a reset pulse for the ID decoder 4. Once the reset pulse is sent to the ID decoder 4, the current code stored in the id decoder is erased. By default, there is no valid code to turn on the sensor, and the sensor unit 1 returns to a sleep state awaiting a new wakeup signal 20. Telemetry transmitter 10 receives its DC power through the reset timer 7, as well as the input signals from the sensor element 8 and the reference sensor element 9. Reference sensor element 9 may be used to calibrate sensor element 8 for environmental effects such as temperature variation, growth in a human or animal subject, arterial and venal cross-sectional changes, temperature expansion in an industrial pipe, and the like.

Telemetry receiver 13 acquires the wireless measurement signal 21 and forwards it on to demodulator 14, which extracts digital data representing the measurement from the modulated carrier signal, as is well known to anyone skilled in the art. The data is forwarded to microprocessor 15 for further processing. Microprocessor 15 converts the data to useable measurement information (for example, a blood pressure measurement) and sends the data to an onboard display unit 16 for either the doctor or patient to read. Microprocessor 15 may also send data to onboard data storage unit 17, which may comprise for example an EEPROM, ROM or RAM. In addition, the data may be sent to communications unit 18 for forwarding the data (for example, either via a PC link or a cell phone link) for remote data gathering. Microprocessor 15 also generates a signal to ID generation unit 12 that feeds into wakeup signal generator 11, for transmission as wake up signal 20 to sensor unit 1.

The embodiment of FIG. 1B can be simplified with the elimination of the ID generation and comparing features, if for example, a single sensor 1 and the scanner 2 make up the entire system. Alternatively, D generation can be eliminated and multiple sensor units 1 can still be used, by employing a unique frequency of telemetry transmitter 10 for each individual sensor unit, as is well known by those skilled in the art.

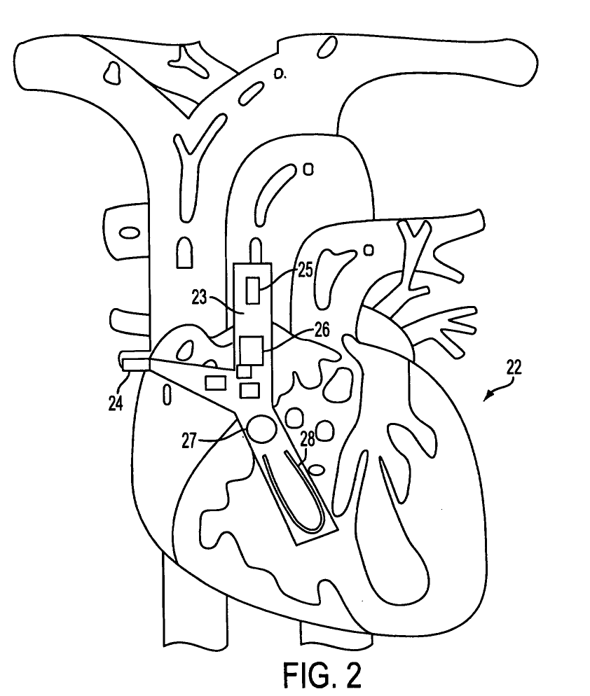

FIG. 2 shows an application employing multiple sensor units for measuring the blood pressure in the heart. It is to be understood that these sensor units can be used and mounted on any appropriate blood vessels, and do not require unique features of the heart for operation. FIG. 2 illustrates an anterior view of a heart 22 with an associated sensor substrate 23 attached therein. Sensor substrate 23 is attached via any medically approved means, (i.e. bio-adhesive, suture, etc.) and is comprised of a flexible and preferably thin substrate material such as KAPTON, silicone, or polyethelene. Flexibility is required so that substrate 23 does not restrict the hearts natural motion. As illustrated, sensor element 24 is attached via substrate 23 at the outside of the inferior pulmonary vein, while second sensor element 25 is attached at the outside of the aorta. Sensor elements 24 and 25 can either be independently constructed devices attached to a flexible substrate 23, or, preferably, may comprise an integrated structure directly embedded in the substrate. Sensor elements 24 and 25 are constructed such that they sense the internal blood pressure of the respective blood vessels, without the need for perforation of the blood vessel. Included on the substrate 23 are electronic components 26 that provide the electrical functions as described for sensor unit 1 in FIG. 1B. These are usually semiconductor chips that are soldered down to the substrate 23. Of course substrate 23 provides the necessary wiring infrastructure to connect all the various electronic components 26 and sensors 24 and 25, as well as battery 27. This “system on board” approach is well known in the semiconductor arts. Additionally, a simple loop antenna 28 is directly integrated in the substrate 23, to allow both for receiving the wakeup signal 20 as well as transmitting the telemetry signal 21.

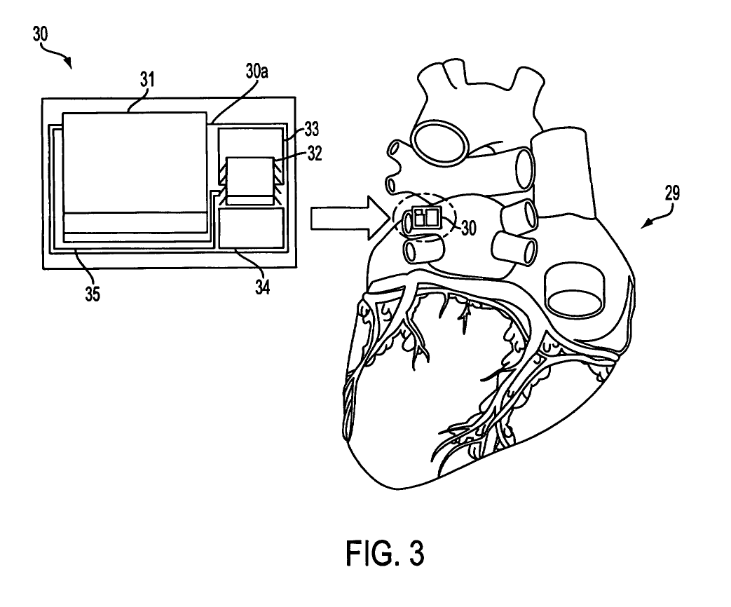

FIG. 3 shows an alternate embodiment for the application of FIG. 2 whereby electronic components 26 of FIG. 2 are reduced to a single ASIC chip 32 in a sensor 30. A flexible substrate 30a in sensor 30 is mounted directly on the superior pulmonary vein as shown in the posterior view of the heart 29. The flexible substrate 30a houses an energy storage device 31 which is either a battery or a self generating power source coupled through the flexible substrate 30a via appropriate wiring to measuring sensor 33 and reference sensor 34 as well as ASIC chip 32. The output from the ASIC 32 is fed to an antenna 35 for full duplex telemetry. Multiple copies of sensor system 30 can be mounted on different combinations of veins and arteries with each sensor system functioning as a fully independent wireless sensor mechanism

FIG. 4A presents a block diagram illustrating another embodiment of sensor unit 1 of FIG. 1A. In the sensor unit 1 of FIG. 4A, an ID number 402 of the sensor unit 1 may be directly embedded within a telemetry signal from the sensor unit to the scanner. This is in marked contrast to the unit described in FIG. 1B, whereby the ID signal is received by the sensor unit 1 as part of the wakeup signal 11. In FIG. 1B, once the unit 1 decodes the correct ID, it wakes up and transmits the requested data without the need for retransmitting the ID back to the scanner. The underlying assumption of operation of the sensor 1 illustrated in FIG. 1B is that sensor 1 will only respond to wakeup signal 11 if its internally recorded ID matches an ID transmitted in wakeup signal 11. In contrast to this approach, the sensor 1 illustrated in FIG. 4A however will broadcast its internally recorded ID with any measurement signal it sends in response to a wakeup request. Hence, in the latter method, multiple sensor units may transmit signals at the same time without interfering with each other. These distinct methods of polling the sensor unit by ID (as in FIG. 1B) and of performing an All-Call where each sensor reports its ID (as in FIG. 4A) are well known in the arts.

The functionality of the embodiment of sensor unit 1 shown in FIG. 4A is further described as follows. A simple, continuous wave 2400 MHz RF signal is detected by the wakeup antenna 404 (¼ wave PCB trace). The signal is fed to an envelope detector 406, which may be realized as a simple matching circuit connected to a diode (for example, Agilent's HSMS-2850). The diode rectifies the 2400 MHz RF signal and a shunt capacitor converts the rectified half-wave signal to a DC level. The DC level is amplified via an operational amplifier 408 (Op Amp) (for example, Maxim IC's MAX409B). The amplified DC level is used to control a Single Pole Single Throw solid state switch 410 which connects an output of battery 412 to the power input of the voltage controlled oscillator (VCO) 414 (for example, Maxim IC's MAX2608) and digital shift register . As long as there is a detected 2400 MHz signal, the battery powers up the VCO 414 and shift register 416 via the SPST switch 410. Hence, the 2400 MHz signal acts as a continuous wakeup for the sensing capability. Once VCO 414 and shift register 416 are turned on, the sensor 418 then measures the distension via a change in resonance frequency. The sensor 418 is an integral part of a frequency-determining resonator of the VCO 414. Any distension of the sensor causes changes in the physical geometry of the sensor 418, which effects the fundamental frequency of the resonator of the VCO 414 and thus the output frequency of the VCO 414. The shift register 416 outputs a serial bit stream that contains the ID of the sensor unit 1, provided by ID number register 402. This bit stream controls the timing of when the switch 422 connects the VCO 414 to the sensor 418 (measurement of distension) or the dummy load 420 (reference frequency). The dummy load 420 provides a reference frequency by which to calibrate out any offsets, temperature effects, and the like. This signal is then fed into matching circuit 424, which matches the impedance of the VCO 414 to that of the PCB Loop antenna 426. The VCO 414 may be operated at 433.92 MHz with the sensor modulating this center frequency by +/−200 KHz. The frequencies indicated above are only illustrative; any frequency combination for wakeup and VCO can be used without loss in generality of function.

We next describe decoding of the wireless measurement signal that would correspond to the input signal 21 of FIG. 1B in greater detail as pertains to the embodiment illustrated in FIG. 4A. Telemetry receiver 13 of FIG. 1B may comprise a standard FM quadrature demodulator such as Philips' SN602A. The receiver 13 demodulates the measurement signal 21 from the sensor unit 1 ( as shown in FIG. 4A), and produces an analog signal proportional to the change in frequency produced by the sensor element 8 and reference (dummy load) 9. This is a typical FM demodulation technique familiar to those skilled in the art. Using this scheme, we are able to obtain both the ID and the sensor data from the same demodulated signal. This is graphically shown in FIG. 4C.

In FIG. 4A, an output 428 of shift register 416 controls the switch 422 between the VCO 414 and the sensor/dummy loads 418, 420. Since the dummy load 420 and the sensor 418 are constructed to resonate at different frequencies, they cause the VCO to produce different output frequencies that are demodulated by the FM quadrature telemetry receiver 13 of FIG. 1B. This variation in resonant frequency translates into different voltage levels on the scanner's demodulated signal, as is the expected function of the quadrature detector. The dummy load 420 will always produce approximately the same voltage level (i.e. its resonant frequency does not change) while the sensor 418 will produce a varying level. The timing for level shifts (as illustrated in FIG. 4C by scanner output 440) is predictable, being governed by how often the VCO 414 of FIG. 4A is switched between the dummy load 420 and the sensor 418, which of course is controlled by the shift register 416 which receives its input as the ID number 402. Hence, by examining the timing pattern of the demodulated signal 440, the board ID 402 may be derived as a function of shift register output 442. In addition, by examining the absolute voltage level of the pulses of output 440, the blood pressure signal (distension) may be recovered since an associated voltage level will be directly proportional to the change in resonant frequency of the sensor 1.

An alternate embodiment of the present invention is illustrated in a sample diagram (FIG. 4B) for the sensor 1 illustrated in FIG. 1B. As earlier emphasized, the sensor 1 of FIG. 4B functions quite differently from the sensor 1 of FIG. 4A. A low power wakeup section consists of three functional blocks, the low duty cycle, low current oscillator 37-43, the wakeup signal receiver 46-50, and the gain stage 44, 45, 51-54. The heart of the low duty cycle oscillator is the uni-junction transistor 39. As illustrated in FIG. 4B, the timing circuit 7 of FIG. 1B comprises a JFET 40 configured as a constant current source set to 400 nA, a programmable uni-junction transistor (PUT) 39, and a reverse biased diode 43 to reduce PUT discharge time. This timing circuit is configured to produce a pulse every second. The constant current source 36 charges up the low leakage 0.1 μF capacitor 42. When the voltage across capacitor 42 equals the firing voltage of the PUT, which is the peak point emitter voltage termed V_{P}, and if the current is large enough, the PUT will enter into the negative resistance region and begin to discharge.

By way of example, in a preferred embodiment of wakeup section 37-43, the maximum firing current required by a 2N6028 PUT 39 for a RG value=1M is 150 nA. RG is the parallel combination of resistors 37 (R3) and 38 (R2). RG=(R2*R3)/(R2+R3). Resistors R2 and R3 set a voltage V_{P}. This voltage is V_{P}=˜(VBat * R2)/(R2+R3). V_{bat} 36, is +3V. Diode 43, which is reversed biased, is used to reduce the PUT discharging time period through the bias lines of amplifiers 44 and 45. When the diode is not used, the discharge period was observed to be about 7-8 milliseconds. With the diode, the discharge time period was reduced to tens of microseconds. The typical duty cycle of this circuit is 0.1%.

The low frequency wakeup signal is a simple On Off Keyed (OOK) modulated 125 Khz signal which is coupled into the implantable sensor via resonant tank circuit consisting of an inductor 46 and a capacitor 47. For approximately 125 KHz resonance, the inductor is 0.4 mH while the capacitor is 4000 pF. The diode pair 48 and 49, serve as rectifiers of the incoming OOK 125 Khz signal while capacitor 50 filters out the carrier of 125 KHz and leaves the demodulated OOK signal. This signal is fed to the non-inverting gain stage consisting of Op Amps 44 and 45 (for example, MAX409B from Maxim) and the associated gain resistors 51-54. The overall gain is typically configured to be 100 so that each stage has a reasonable gain of 10.

The OOK signal, which now represents the desired ID of the implantable sensor, is fed into the shift register 55 which acts as a serial to parallel converter. The parallel output of the shift register is fed into the ID comparator 56 which compares the OOK signal to the onboard hardwired ID 57. If the OOK signal matches the hardwired ID 57 on a bit to bit comparison, then the ID comparator will send out a low to high transition on the OUT pin. This signal controls the solid state switch 58. The switch, when activated by this control signal, connects the battery 36 to the remaining components of the circuit; the reset timer, sensors, and telemetry circuits.

When switch 58 is activated, it supplies power to the timer circuit 59 and the divide by N counter 60. The timer circuit output is split between the divide by N counter 60 and switch 71. The output of the divide by N counter 60 is fed to the base of an NPN transistor 61. With no signal present at the base of the transistor 61, pull up resistor 62 allows for the battery to place a “HIGH” signal on the clr pins of the shift register 55 and the ID comparator 56, allowing them to operate. As soon as the base of the transistor 61 is activated, the clr pins of both digital devices go “LOW” and they both shut down thereby invalidating any current OOK signal, turning off switch 58 and sending the implantable sensor back to sleep until the next OOK valid signal is received.

The measuring sensing element 8 of FIG. 1B is a simple relaxation oscillator consisting of two hex inverters 63 and 64, a timing resistor 65 and the sensor (which is equivalent to a variable capacitor 66). As the variable capacitor 66 changes its capacitance in accordance with the change in blood pressure within the blood vessel, the frequency of the oscillator changes according to F=1/(1.8 R*C). It is to be understood, that an alternative construction of the oscillator would allow for a fixed capacitor, and a variable resistor that functions as the measuring element, i.e. using a resistive strain gage on a flexible substrate that surrounds the blood vessel, should such a sensing element exist.

The reference relaxation oscillator 9 of FIG. 1B consisting of components 67-70 performs similarly, except that the capacitor 70 is fixed and not variable (i.e., it does not measure the blood pressure but is still subjected to the same environment as the variable capacitor). The timer 59 controls the switch 71 which injects this oscillation signal of the respective measuring and reference oscillators, into the phase locked loop (PLL) 73-76, at the appropriate location within the loop, to be sent to the scanner via antenna 77. The PLL 73-76 is a standard configuration consisting of crystal reference 74, phase detector 75, divide by N counter 76, voltage-controlled oscillator (VCO) 72, and loop filter 73. Those skilled in the art will readily identify these components, as PLL's are well known in the art. In essence, the PLL 73-76 is set at a center frequency of approximately 400 MHz and sends out an FM modulated signal with the modulation being the alternating frequency of the sensing oscillator 63-66 and the reference oscillator 67-70. The scanner 2 of FIG. 1B then receives this FM signal, demodulates it, compares the reference signal to the measuring signal, and continues to process the signal accordingly. The construction of such FM receivers is well known in the art.

It is important to note that even though the demodulation of the FM signal can be done by the same quadrature detector as referenced in the detailed explanation of FIG. 4A, the demodulated analog voltage signal contains drastically different information for each of the embodiments of FIGS. 4A and 4B. If the sensor is that of FIG. 4A, the demodulated signal will contain both the ID and the Sensor data, while if the sensor is that of FIG. 4B, only the sensor data is contained within the demodulated signal. There is no need for the ID to be transmitted for the sensor of FIG. 4B, as only the correctly polled sensor will respond. Since the scanner knows which ID it queries, it knows that only the right sensor has responded.

Consequently, the wakeup signal 20 for scanner 2 in FIG. 1B differs pending on the implementation of FIGS. 4A,B as the sensor of choice. The embodiment of FIG. 4A requires the wakeup signal 20 to consist of a pure continuous wave RF signal ( simple carrier wave) with no ID information embedded in it, while the embodiment of FIG. 4B requires the wakeup signal 20 to be a modulated RF signal with the sensor ID embedded as the modulated data.

Clearly, certain of the components in FIG. 4B can be eliminated in order to streamline and limit specific functions of this preferred embodiment without loss of general overall functionality. For example, the ID comparator 56, and all associated circuitry such as the timer 59 can be removed so that the device functions only when the wakeup signal is continuously present. Additionally, the reference oscillator 68-70 can also be removed if one desires to implement this embodiment as a free running device without the benefit of reference.

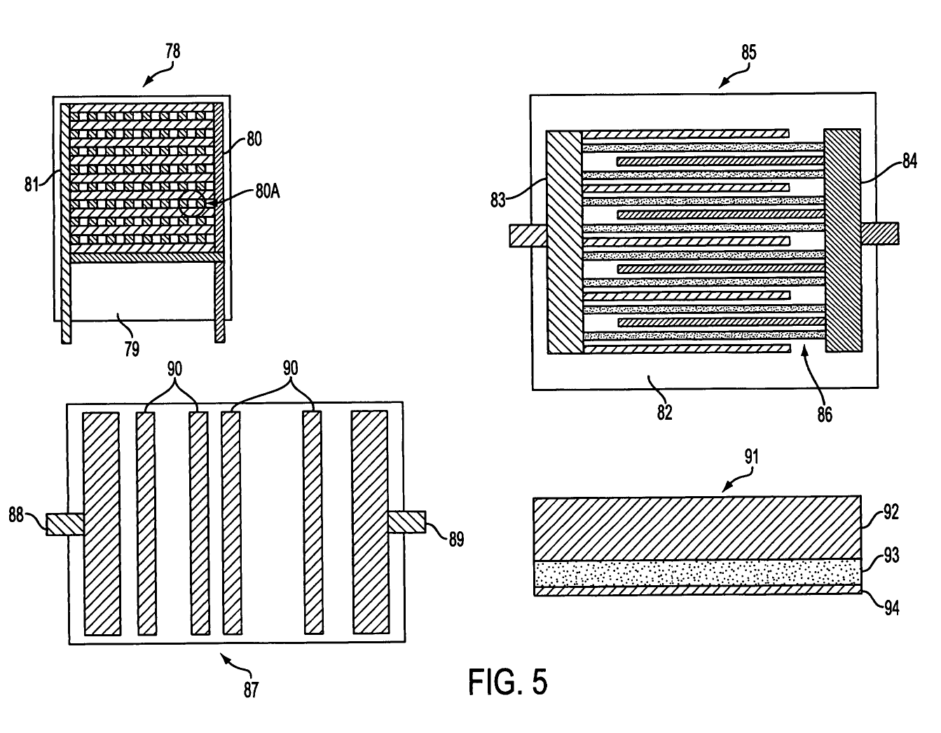

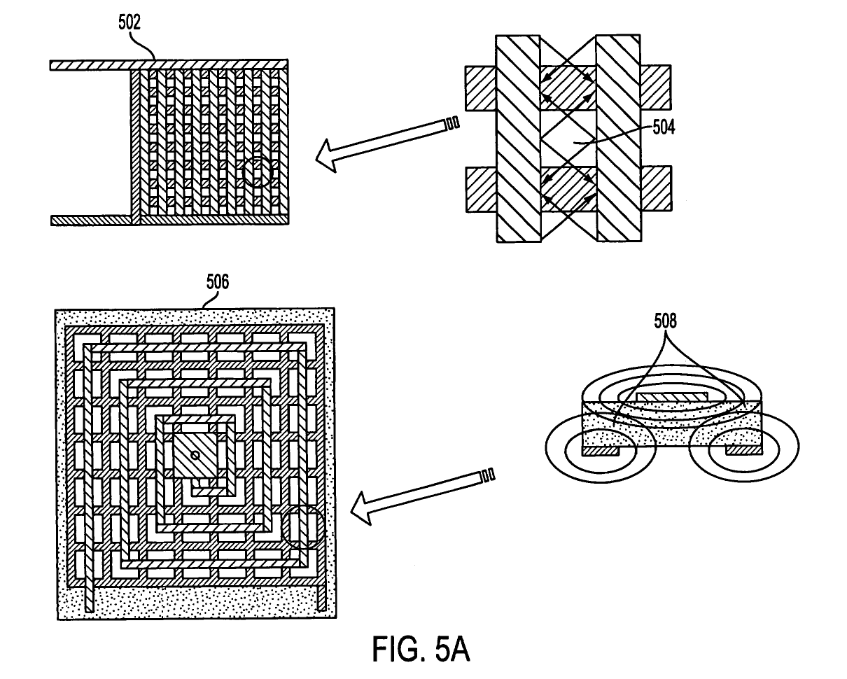

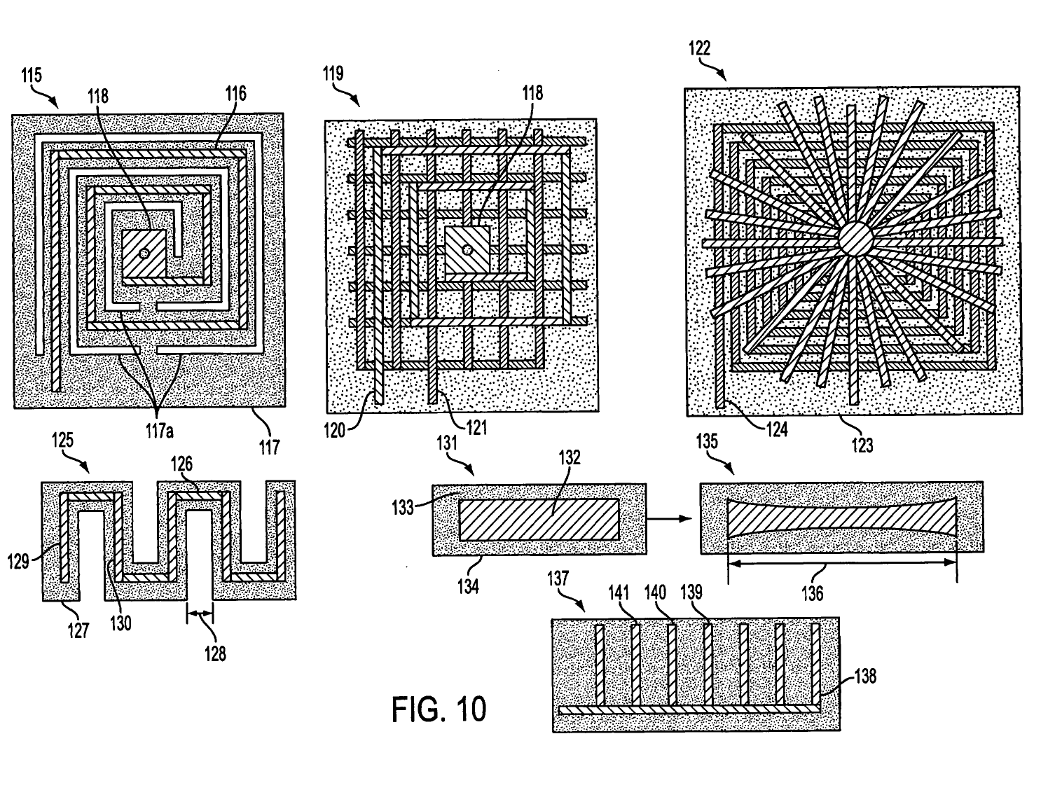

FIG. 5 illustrates a number of different sensor configurations that can be used as both the sensing and reference capacitor elements 66 and 70 respectively. It is by no means exhaustive, and is intended to illustrate the wide variety of capacitive sensing element topologies contemplated by the present invention. Screen capacitive sensor 78 includes an upper electrode 81, a lower electrode 80 and an intervening flexible substrate 79. The sensor 78 is constructed as a thin monolithic structure with the upper and lower electrodes patterned copper, electrodeposited onto 0.002″ thick KAPTON which serves as the flexible substrate 79. The sensor 78 may be wrapped around a blood vessel, for example, and attached appropriately. As the blood vessel expands and contracts due to internal blood pressure, and fringing fields generated by electrodes 80, 81 in the non overlapping regions 80A distort due to the change in curvature of the blood vessel, and thereby sensor 78. This change in fringing fields manifests itself as a change in overall capacitance of the device, which allows for measuring the change in blood pressure. This effect is illustrated, for example, in FIG. 5A by fringing fields 504 associated with screen capactive sensor 502. Alternatively, inductive fringe fields 508 are illustrated for inductive sensor embodiment 506, which is described in further detail as sensor 119 of FIG. 10.

Several additional embodiments for a capacitive sensor are illustrated in FIG. 5. Like capacitor 78, interdigitated capacitor 85 has a monilithic structure, but has both electrodes 83 and 84 residing on the same side of flexible substrate 82. Again, $he electrodes may be patterned copper on 0.002″ KAPTON. A variation in capacitance is achieved by having slits 86 that are cut through the KAPTON substrate between the fingers of the interdigitated capacitor 85. The slits 86 allow the individual fingers of electrodes 83, 84 to move as the blood vessel expands and contracts. As the fingers separate from each other or come closer to each other, the overall capacitance of the structure changes.

Alternatively, one may use a SAW (surface acoustic wave) device 87 mounted on a flexible substrate to provide the variation in capacitance needed to make the measurement. As the fingers of the resonant device 90 positioned between electrodes 88, 89 are pulled or pushed apart, due to the flexibility of the substrate and its distortion due to the expansion or contraction of the blood vessel, the resonance frequency of device 87, represented by a combination of inductance and capacitance, changes accordingly.

Additionally, as shown in the cross-sectional view 91, a simple capacitive structure consisting of both upper and lower electrodes 92 and 94, with an intervening layer of elastomer 93 can be used as the measuring device. If the upper electrode is sufficiently thick so that it is less compliant than the elastomer, as the blood vessel contracts or expands, the elastomer will compress or expand, thereby changing the capacitance of the structure as the upper and lower electrode change their separation distance.

There are many other topologies that are suitable for use in sensor element 8 in the sensor unit 1 of FIG. 1B. Furthermore, as the underlying physical sensing principle is based on an electromagnetic effect in changing either the components' inductance, capacitance and/or resonant frequency, the topologies are all geometry based, and are not dependent to first order, on material parameters. For example any of the popular biocompatible materials such as gold, titanium, niobium, TEFLON, KAPTON, PVC, Polyethylene, and the like can be used as the conductors and dielectrics used in the sensor topology chosen. Additionally, as the underlying physical principle is geometrically dependent, size scalability as a function of frequency of operation is entirely at the device designer's discretion.

FIG. 10 illustrates several sensor element topologies that are applicable in addition to the topologies of FIG. 5. All designs can be used in either low frequency operation (whereby the structure needs to be resonated with the associative inductor or capacitor), or in high frequency where the devices described are a substantial fraction of a wavelength and thereby can exhibit self-resonant characteristics. All devices function in the aforementioned prescribed manner of changing their resonant frequency (inclusive of the associative inductance or capacitance in low frequency, or self-resonant in high frequency) as a function of the change in distension to pressure applied to the blood vessel.

Sensor structure 115 includes a spiral inductor 116 over a ground plane 117 with slits 117a cut, and a plated through hole 118 connecting the spiral inductor to the ground plane 117, and functions in a similar manner to the interdigitated capacitor 85 of FIG. 5. However, in this case 115, the distension causes the arms of spiral inductor 116 to move positionally due to the slits 117a, thereby changing the inductance of the device. An appropriate value of capacitance must be added in order to resonate the structure 115.

Sensor structure 119 is a combination including a spiral inductor 120 as the top electrode and screen sensor 121 serving as the bottom or ground electrode. Again, plated through hole 118 connects the spiral inductor to the screen sensor ground plane. Because the ground plane is discrete and not continuous as in structure 115, slits are not required to allow a change in inductance due to distension, and a complete monolithic changing inductance structure is realized. As distension occurs, the fringing fields “fill in” the ground plane to a varying degree dependent on the distension, thereby changing the inductance of the overall structure. As earlier noted, magnetic fringe fields 508 are illustrated in association with inductive sensor 506 in FIG. 5A.

In sensor structure 122, a top electrode 123 is arranged with radial arms overlapping bottom electrode 124. It is to be noted that this structure 122, will have the greatest sensitivity to pressures exerted to the center of the top electrode, thereby making this structure suitable for mounting as a diaphragm structure rather than as a structure surrounding a blood vessel. Accordingly, sensor structure 122 is particularly suitable for measuring distension of surfaces of bendable non-tubular members such as flat diaphragms. For example, it is contemplated to place this structure on the tip of a catheter, whereby the structure acts as a pressure-sensing diaphragm. Given that clinical catheters rarely exceed 10 French in size, this structure is contemplated to be used as a discrete capacitor resonated with a discrete inductor, rather than a self-resonating structure, as self resonance would occur in the millimeter wave frequency range.

Other related sensor geometries are clearly suggested by structure 122 as well. Identical functionality is obtained for example, if a bottom electrode is realized in a radial star pattern while a top electrode is realized in a spiral, allowing for implementation at the closed end of a flexible tube rather than along the circumference of the tube. The areas of overlap produce the capacitor and the adjacent metalization produces the fringing fields.

Sensor structure 125, is that of a microstrip meander line that consists of upper electrode 126, separated from a dielectric material, with ground plane 127 serving as lower electrode. This is a distributed structure such that the length of the meander line represents ½ wavelength at the frequency of operation. The meander line possesses some resonant frequency. As the structure 125 is attached to the blood vessel that distends, the gaps 128 will widen accordingly. This widening in turn reduces the coupling between the meander line arms 129 and 130, thereby altering the resonant frequency.

Sensor structure 131 is a microstrip ½ wavelength resonator consisting of upper electrode 132, flexible and compressible dielectric 133 (for example, comprising latex, butynal, silicone, and the like) and lower electrode 134. As the blood vessel distends the physical structure is stretched thereby lengthening the upper electrode. The new electrode length 136 now gives rise to a lower resonant frequency. Hence the distension of the blood vessel directly produces a change in resonant frequency as is taught in this patent application.

Sensor structure 137 is somewhat similar to structure 125. Upper electrode 138 is arranged with a multiplicity of combs. The individual combs can be ¼wavelength in length in order to realize resonant behavior. Comblines 139, 140, 141 all couple to each other to produce an overall resonant effect. As the blood vessel distends, this coupling will vary due to the curvelinear change in a coupling coefficient which will directly effect the resonant frequency. For example, if we focus on the coupling of all comblines to combline 139, it is clear that the coupling of combline 141 and 139 will be greatly effected by the arclength between them. This effect can be extrapolated without loss in generality to any combination of comblines, and therefore the change in resonant frequency represents the aggregate of change in coupling coefficients within the structure.

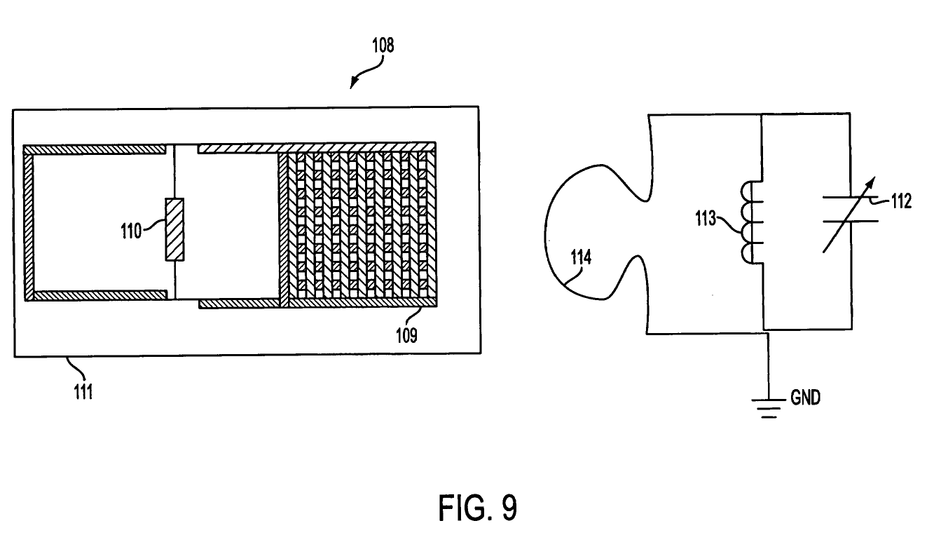

It is well understood by those skilled in the microwave and radio engineering arts that each of the aforementioned structures can then be embedded in electronic circuits whereby the change in resonance affects an electronic signal. For example, structure 125 can be used as the frequency selective element for either a serial or parallel feedback oscillator. This oscillator in turn can be substituted for components 63-66 in FIG. 4B with no loss in general functionality. Alternatively, the structures may be substituted in structure 108 of FIG. 9 in order to realize a completely passive transducer. An even more clever arrangement allows for structures 125 or 137 to be directly combined into a self-antenna arrangement whereby the loop antenna 111 in structure 108 is directly combined as part of the resonant structure itself. Alternatively, these structures can be embedded as tunable components in filter structures such that the filters exhibit frequency selectivity based upon blood vessel distension.

It is also of importance to realize that the topologies presented here can all be stacked into 3D structures in order to maximize the sensitivity of the overall sensor. For example, multiple copies of structure 122 can be stacked one on top of the other and electrically connected in parallel so that the overall effect of the distension of either the diaphragm or flexible tube effects each copy in a uniform manner and the overall change in capacitance is a geometric sum of all the individual sensors. Clearly, as the flexible structure 122 is made more compliant than the bending surface, many copies of the sensor can be stacked together without fear of compromising the natural bending motion of the measured surface.

Packaging of electronics for long-term placement within a biological host such as a human being has always been of major concern in the development of implantable medical devices. Many techniques for hermetically sealing the electronics have been disclosed. It is clear that the most effective way to hermetically seal electronics is to completely encase the electronics in some impermeable casing such as biocompatible titanium or ceramics. However, by completely encasing the electronics, one cannot access the electronic signal for which the device was created. In essence one can easily solve the long-term biocompatability issues at the expense of having a completely useless device. Much work has gone into the ability to extract useful signals from the encased electronics via hermetic feedthroughs, arrays of feedthroughs, and the like. However, using sensor topologies as taught herein, the need for feedthroughs are eliminated and all the electronics components can be completely, hermetically sealed in ceramic/metal packages. Since all the sensors taught herein do not require physical contact between the surrounding electronics and the sensor, we exploit this operational condition in order to realize packaging of the device for long-term implantation. By coupling to the sensor either through inductive or capacitive means, the electronics such as batteries, transistors, IC's etc. can all be isolated in a hermetically sealed package, while the interface with the sensor is done through an electromagnetically permeable substrate.

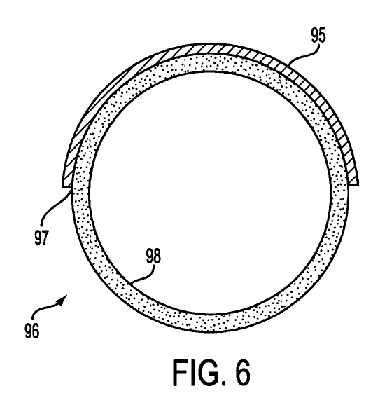

FIG. 6 indicates a cross-sectional view of the mounting 96 of the sensor 95 on the blood vessel 98. The sensor may be any of the topologies as indicated in FIGS. 5 and 10. The sensor may be attached to blood vessel 98 via a bioadhesive 97 applied between sensor 95 and blood vessel 98, or may alternately be held in place via a standard suturing process.

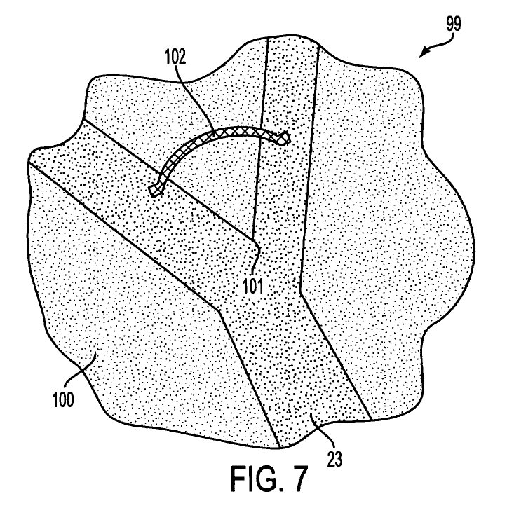

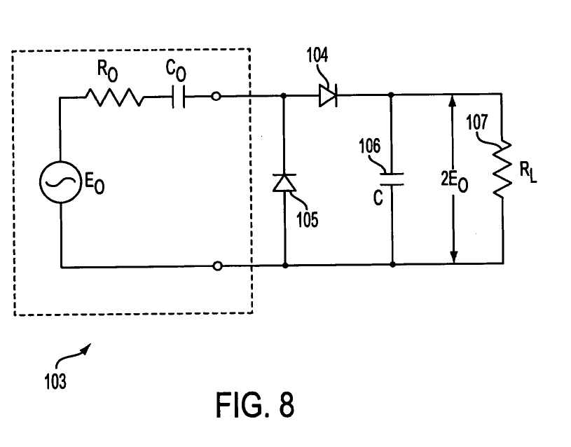

An alternative power generation arrangement to battery 36 of FIG. 4B is illustrated in FIG. 7. In FIG. 7, a partial anterior view of the heart 99 shows the previously taught flexible substrate 23 of FIG. 2 attached in a region of the “Y” split 101 mounted on the heart 100. A wire or strip 102 made out of a piezoelectric material such as PZT or PVDF is mounted between the arms of the “Y”. As the heart contracts the wire 102 is flexed thereby generating a charge due to the piezoelectric effect. This charge can then be used, for example, as illustrated in FIG. 8. In FIG. 8, piezoelectric wire or strip 102 is represented by an equivalent circuit 103 consisting of the voltage generator Eo, internal resistor Ro and internal capacitor Co. Circuit 103 generates a voltage which is rectified by the diodes 104 and 105 with capacitor 106 storing the charge. The capacitor may serve as the implantable sensors own direct battery source provided it is sufficiently large (for example, surface mount AVX4444 47uF capacitor made by American Technical Ceramics) or alternatively may be used to supply a trickle charge to an onboard rechargeable battery 36 via generic load resistor 107.

FIG. 9 indicates a view of a passive transducer 109 that is suitable for implantation via catheterization. Transducer 108 is powered by radiated RF from the wakeup/signal 20 of FIG. 1B as is routinely taught in the radio frequency identification (RFID) arts. In the embodiment of FIG. 9, the screen sensor of FIG. 78 of FIG. 5 serves as a variable capacitor 109 that varies the capacitance with the distension of the blood vessel. A fixed inductor 110 is used to resonate the variable capacitor 109 such that, when the blood vessel distends, the overall resonance frequency shifts. Loop antenna 111 is used to couple in the RF energy from signal 20, and to re-radiate the energy as signal 21 of FIG. 1B. Loop antenna 111 is designed to allow for the full deviation of the frequency shift of the resonant circuit to be contained within the useful bandwidth of the antenna. The equivalent circuit is shown as a combination of variable capacitor 112, inductor 113, and loop antenna 114. The wakeup transceiver 11 of FIG. 1B sends out a variable frequency in a scanning fashion and looks via telemetry receiver 13 for a return signal 21 from sensor unit 1. As only the resonant frequency of the transducer 109 will provide a return signal that the receiver 13 will pick up, a change in resonant frequency of the returned signals from sensor unit 1 can be correlated to an implied blood pressure within the blood vessel.

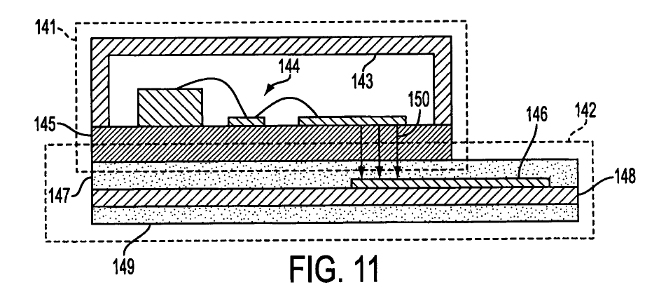

FIG. 11 presents a cross-sectional view of an embodiment of the present invention with hermetically sealed electronics in one integrated sensor/transducer device package. The electronics hermetically sealed package 141 consists of a cover 143 that is hermetically sealed to a dielectric substrate 145. Contained within this package are the electronics 144. The hermetically sealed package may be realized for example by having the cover 143 made of titanium while the substrate is made of titanium coated alumina, so that a metal to metal bond is effected. Alternatively, the substrate might be glass and an anodic bonding with the either ceramic or titanium cover 143 provides the hermeticity. Those skilled in the electronic packaging arts will immediately realize the many other combinations of materials and bonding topologies that can be used for hermetically sealing the electronics. For capacitively coupling the electronics 144 to the sensor 146 through the electric field 150, sensor 146 is proximally located to the electronics, except it is separated form direct contact with the electronics by a dielectric substrate 147 of the hermetic package. The sensor itself can be packaged in its own hermetic package 142 consisting of upper 147 and lower 149 dielectric materials such as PVC, TFE, or polyethylene, that completely surround the sensor substrate 148. Substrate 148 as taught herein could be made, for example, of KAPTON polyamide material.

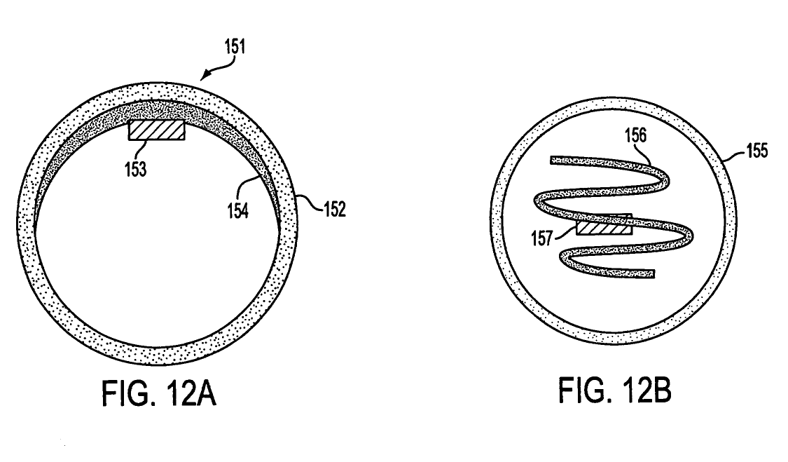

The sensors taught herein can also work equally as well, for example, attached to the inside of the blood vessel. This internal sensor functions identically to the aforementioned external sensor using the same topologies. FIG. 12a shows a cross-sectional view of an internal sensor unit 151, positioned internally to blood vessel 152 and consisting of hermetically sealed chip 153, as taught above employing capacitive or inductive coupling, and flexible sensor 154. The sensor/unit 151 is attached to the blood vessel as previously described in conjunction with FIG. 6. The internal sensor may be delivered to the appropriate location, for example, via catheterization. FIG. 12(b) shows a cross-sectional view of flexible sensor 156, along with hermetic chip 157, rolled up in order to fit within the catheter 155 used to deliver the sensor/transducer to its final location. As the sensor is pushed out of the catheter, it unfurls and is attached to the inner wall of the blood vessel as shown in FIG. 12(a). Because of miniaturization and power constraints, a completely passive sensor such as taught in FIG. 9 would be a preferred embodiment for such internal sensors. Alternatively, self-powering techniques, such as use of PZT material, as taught in FIG. 7, may be employed.

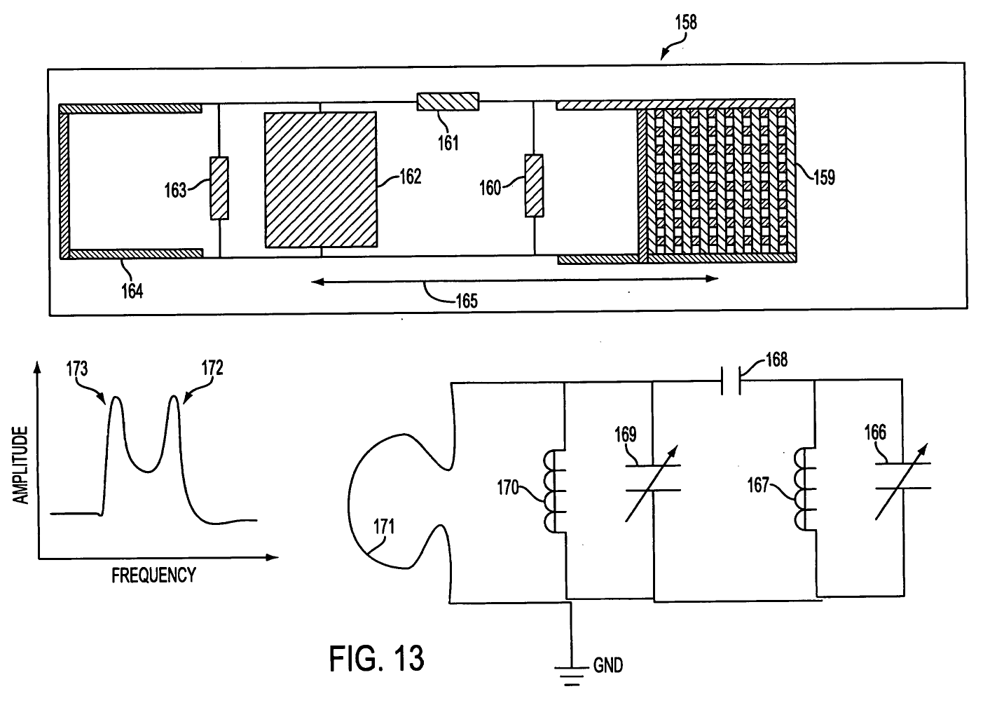

The use of an internal sensor also suggests the possibility of integrating two sensors that together provide both pressure measurement and cardiac stroke volume measurement within the same device. This is because the internal sensor can measure pressure directly, since it is in the blood stream, as well as the radius of curvature of the blood vessel as it distends. FIG. 13 illustrates a sensor/unit 158. A first sensor element includes screen sensor 159 and an associated inductor 160 to form a resonator. A second sensor element includes sensor 162 (which is a plan view of the compressible sensor 91 as taught in FIG. 5, and associated inductor 163. Both sensors are separated by a capacitor 161, which is electrically in series between the two sensors. Antenna loop 164 allows for energy to be coupled in and out of the sensor/unit 158. The first and second sensor elements are separated by a known physical distance 165. The capacitor 161 effectively de-couples the two resonators from each other so that examination of the frequency vs. amplitude plot of FIG. 13 reveals a distinct resonance peak 172 for the first sensor element and a distinct resonance peak 173 for the second sensor element. The separation of peaks 172, 173 reflects the distance 165 between sensor elements. An equivalent circuit for sensor/ transducer 158 is indicated by the dual tank circuits 166-167 and 169-170 with the variable capacitors acting as the sensors.

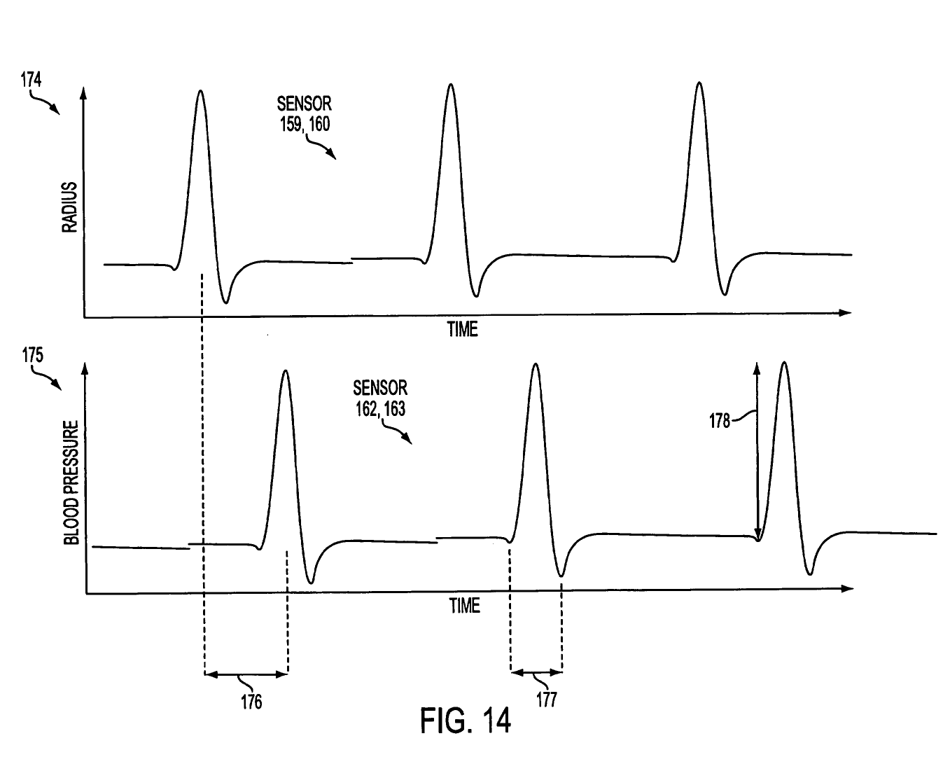

The operation of the sensor unit 158 is further explained as follows, and is illustrated in FIG. 14. Sensor 159, 160 of FIG. 13 generates a radius change waveform given by the time versus radius graph 174 of FIG. 14. Sensor 159, 160 is used in this embodiment, for example, to measure the radius of the blood vessel. As taught above, the sensor topologies primarily measure the distension of the blood vessel. We are then able to correlate the distension with the pressure. However, the sensor directly measures the distension. Knowing the initial radius of the blood vessel, which can be measured at the time of implantation, as the blood vessel distends due to the increase in blood pressure the radius of the blood vessel changes accordingly. Hence, a change in frequency of sensor 159, 160 is directly related to the change in radius of the blood vessel. Sensor 162, 163 of FIG. 13, the compressible sensor, generates a blood pressure versus time waveform 175. Sensor 162, 163 measures the blood pressure directly as the compression of the sensor is directly proportional to the pressure applied. The height of peak 178 is the measure of the peak blood pressure. At this point we have directly measured the pressure P and the radius r of the blood vessel. As mentioned earlier, sensor 159, 160 and sensor 162, 163 are separated by a known distance 165. This distance manifests itself as a delay in the blood pressure peaks between 174 and 175 and is indicated as the delay time 176, since the pressure wave takes a finite amount of time to traverse the distance 165. Since the distance 165 is fixed and the delay time 176 is known we can calculate the velocity of the blood flow as velocity=distance/ time. Additionally, from examination of the blood pressure waveform 175, the duration of the stroke or pressure pulse is also known, 177. As such, all measurands needed to determine both the blood pressure and the stroke volume are now known. The volume of blood per stroke can be easily calculated as

Volume=II* r² * velocity * stroke time [1]

Where r can be derived from the resulting waveform 174, velocity is determined from delay 176, and distance 165, and stroke time is given by 177. Cardiac output is (Volume/stroke) is easily determined from the above equation.

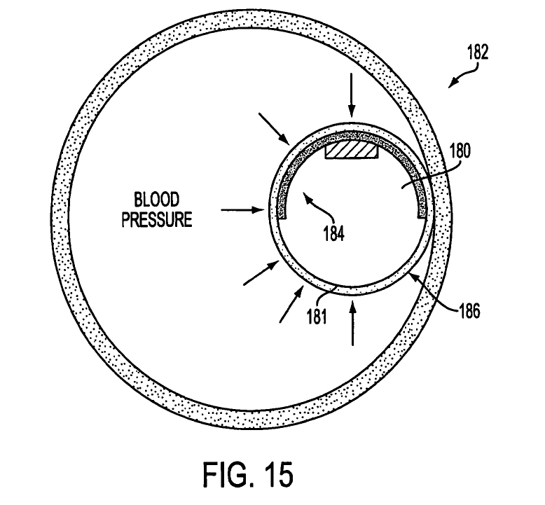

Another packaging scheme contemplated by the present invention is that of enclosing the entire electronics and sensor in a hermetic dielectric (quartz, boron silicate, ceramic, etc.) material capsule and allowing the sensor to measure the distension of the capsule rather than the actual blood vessel. The capsule then can be delivered via catheterization and anchored to the blood vessel walls, promoting tissue growth, and affecting a permanent bond to the blood vessel. A cross-sectional view of this approach is shown in FIG. 15, where a circular cross section for the capsule 180 has been assumed for clarity. Capsule 180 is positioned within blood vessel 182, where sensor unit 184 is attached to an inner surface 181 of capsule wall 186. Pressure changes within blood vessel 182 cause distension of capsule wall 186, which can be measured by sensor unit 184. Other capsule shapes such as corrugated circular, dumbbell, and the like, that amplify the mechanical distension of the capsule may be chosen to form the capsule.

As should be appreciated by those skilled in the art from the foregoing, the subject invention provides various advantages over the prior art, particularly for applications requiring blood pressure determination, cardiac output determination and/or monitoring over extended periods of time. For example, with the subject invention measurements may be made continuously without disturbing the patient after the initial index surgery of applying the device, the device functions independent of the patient's activities. Moreover, the system can provide better accuracy than standard cuff methods as it readily measures the blood pressure directly in proximity to the heart. Further still, since the system of this invention is capable of providing data whenever required, (for example, periodically or non-periodically over an extended period of time), the system can provide better data for the management of various conditions, (for example, high blood pressure, congestive heart failure, heart drug dosage), in patients.

The subject system can be used to reduce the frequency of visits to a health care provider for blood pressure determinations. In fact, since a portion of the system is implanted, while the other portion is readily transportable and able to communicate with other devices (for example, cell phones), the need for visits to a health care provider's office for a pressure determination can be reduced or eliminated entirely. For example, the system of this invention may be used in the workplace to routinely gather data while the subject is at work.

Moreover, in the externally attached sensor embodiments of the present invention, since the artery is not punctured or otherwise penetrated by the sensor/transducer, the risk of a blood-born infection or other adverse effects on the patient is minimized, if not eliminated.

As should be appreciated by those skilled in the art, the treatment of various diseases or physiological conditions may greatly benefit from the acquisition of reliable data indicative of a person's blood pressure taken over an extended period of time. Data regarding other physiological factors, such as the patient's temperature, heart rate, muscle tension, sleep patterns, perspiration, and tremors, if correlated to the monitored blood pressure are likely to provide additional information facilitating the diagnosis and/or treatment of diseases or physiological conditions. Furthermore, with continued data collection, data patterns may emerge which will serve as event predictors such as high blood pressure episodes or even the onset of heart attacks. Moreover, various environmental factors, such as the time of the measurement, the ambient noise, ambient temperature, ambient light, air movement, etc., may also play a role in a person's blood pressure. Thus, it is contemplated that the subject invention be used in a system monitoring one or more of the foregoing physiological and/or environmental actors, whereupon the data regarding the patient's blood pressure and one or more of the other factors may be correlated to provide valuable information from which a diagnosis or treatment may be developed. Further still, the system may make use of various alarms or other means to indicate when one or more predetermined factors has been exceeded.

It should be noted that while the subject invention has been discussed with reference to monitoring/determining the subject's blood pressure, particularly at the heart, the teachings of this invention can be implemented to determine/monitor other parameters or fluids flowing through vessels, ducts, lumens in the body of a living being, or industrial process, providing that such parameters can be calculated or determined in response to the position of an unconstrained portion of the wall of the vessel, duct or lumen.

Various alternative system embodiments may be considered as well. For example, other schemes for overlaying an ID on the pressure signal may also be used. One such scheme is to heterodyne the ID with a mixer whereby the sensor signal provides the local oscillator.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

What is claimed is:

1. A system for remotely sensing distension or bending in a member, the system comprising a sensor, the sensor comprising:
a sensing element for sensing a measure of distension or bending in the member, wherein at least a portion of the sensor is in physical contact with the member and is sufficiently flexible so that the geometry of the member and distension or bending in the contacted portion of the member remain substantially unaffected by the sensor;
a response circuit for generating a reply signal indicating a measured distention or bending sensed by the sensing element; and
a radiating element for wirelessly transmitting the reply signal.

2. The system of claim 1, wherein the response circuit comprises a transmitter, the transmitter comprising at least one of a voltage controlled oscillator (VCO) and a phase locked loop (PLL).

3. The system of claim 1, wherein the sensor further comprises a receiver for receiving a wireless request signal for sensing.

4. The system of claim 1, wherein the sensor further comprises a power source.

5. The system of claim 3, wherein the receiver further comprises a wake-up circuit for causing the receiver to periodically prepare to receive a request signal.

6. The system of claim 3, wherein the receiver further comprises a decoder for verifying that the request signal is directed to the sensor.

7. The system of claim 6, wherein the receiver verifies the request signal by comparing information in the signal to a unique identifier for the sensor.

8. The system of claim 6, wherein the receiver further comprises a reset timer for resetting the decoder and decoupling power from the sensing element and the transmitter.

9. The system of claim 4, wherein the power source is a battery.

10. The system of claim 4, wherein the power source is a self-generating power source for converting mechanical energy to electrical energy.

11. The system of claim 10, wherein the source of mechanical energy is mechanical motion, and the source of the mechanical motion is associated with one of a group of sources including blood vessels, organs, living beings and industrial devices and machinery.

12. The system of claim 10, wherein the power sources comprises a piezoelectric element.

13. The system of claim 4, wherein the power source is an eternally-supplied radio frequency (RF) signal and wherein the response circuit comprises a resonant transducer in which the sensing element operates as one of a variable capacitor and a variable inductor, the response circuit passively producing the reply signal in response to the externally-supplied RF signal.

14. The system of claim 13, wherein the sensing element further operates as the antenna

15. The system of claim 1, wherein the sensing element signals measures distension or bending as a change in an electrical property of the element, the electrical property being selected from the group consisting of capacitance, inductance, resistance, frequency, phase and amplitude.

16. The system of claim 1, comprising a plurality of sensing elements.

17. The system of claim 16, wherein ones of the plurality of sensing elements are stacked to comprise a multi-element sensor.

18. The system of claim 1, comprising a plurality of sensors, wherein each of the plurality of sensors has a unique identifier.

19. The system of claim 1, further comprising a reference element for calibrating the sensing element.

20. The system of claim 1, wherein the sensor is hermetically sealed in a single package flexibly mounted to the member.

21. The system of claim 20, wherein the package comprises materials suitable for implanting within a biological entity.

22. The system of claim 21, wherein the package materials are selected from the group consisting of gold, titanium, titanium-coated alumina, ceramics, PVC, TFE, polyethylene and KAPTON.

23. The system of claim 1, wherein the sensing element is separately packaged from the other elements of the sensor.

24. The system of claim 1, wherein the member selected from the group consisting of flexible tubes, bending plates, beams and columns.

25. The system of claim 24, wherein the sensing element is configured to be conformally affixed to a surface of a flexible tube.

26. The system of claim 25, wherein the sensing element is configured to be affixed to the tube surface by means of a bioadhesive.

27. The system of claim 25, wherein the sensing element is configured to be sutured to the surface of the tube.

28. The system of claim 25, wherein the sensing element is configured to be affixed to an interior surface of the flexible tube.

29. The system of claim 20, wherein the sensing element is foldable for delivery to the interior of the tube by catheterization.

30. The system of claim 24, wherein the sensing element is configured to be affixed to an exterior surface of the flexible tube.

31. The system of claim 24, wherein the sensing element is affixed to an interior surface of a capsule, the capsule itself being a flexible member and being configured for placement within an interior volume of the flexible tube.

32. The system of claim 31, wherein the capsule comprises a hermetically sealed dielectric material.

33. The system of claim 15, wherein the sensing element comprises a pair of planar electrodes positioned on a flexible substrate.

34. The system of claim 33, wherein the flexible substrate comprises one or more materials that are selected from the group consisting of KAPTON, elastomer, silicone, polyethelene and any other suitable thin, flexible material.

35. The system of claim 33, wherein the sensing element comprises a pair of planar screen electrodes each positioned on an opposing surface of the substrate, such that electrode members in one screen electrode are oriented at an angle with respect to electrode members on the other screen electrode so that fringing fields are formed on the substrate at positions where electrode members of the first electrode and electrode members of the second electrode are non-overlapping.

36. The system of claim 33, wherein each of the pair of planar electrodes of the sensing element has a plurality of fingers and is positioned on a surface of the substrate such that fingers in each of the pluralities are interdigitated, and wherein portions of the substrate between interdigitated fingers are relieved to facilitate movement between the interdigitated fingers.

37. The system of claim 33, wherein the sensing element comprises a surface acoustic wave device comprising a pair of planar electrodes having parallel surfaces positioned on a surface of the substrate at a known distance apart, and further comprising a plurality of planar fingers interposed on the substrate surface at known positions between the electrodes and oriented in parallel with the electrodes.

38. The system of claim 33, wherein the sensing element comprises a pair of planar electrodes each positioned on an opposing surface of the substrate, and one of the pair of electrodes and the substrate are each substantially more compliant than the other electrode.

39. The system of claim 33, wherein each of the pair of electrodes is positioned on an opposing surface of the substrate, a first of the pair of electrodes comprises a ground plane and a second of the pair of electrodes comprises a spiral conductor electrically connected to the ground plane.

40. The system of claim 39, wherein and the substrate is relieved at points intermediate to adjacent portions of the spiral conductor.

41. The system of claim 39, wherein the first of the pair of electrodes comprising a ground plane is a screen electrode.

42. The system of claim 39, wherein the first of the pair of electrodes comprises radial portions radiating from a position adjacent to a center of the second spiral inductor.

43. The system of claim 33, wherein a first of the pair of electrodes comprises a meander line having a length related to a fractional wavelength of an operating frequency for the sensing element and the second of the pair of electrodes comprises a ground plane, the first and second electrodes being positioned on opposing surfaces of the substrate.

44. The system of claim 33, wherein a first of the pair of electrodes is a flexible microstrip electrode having a length related to a fractional wavelength of an operating frequency and the second of the pair of electrodes comprises a ground plane, the first and second electrodes being positioned on opposing surfaces of the substrate.

45. The system of claim 33, wherein a first of the pair of electrodes comprises a comb having tines of a length related to a fractional wavelength of an operating frequency and the second of the pair of electrodes comprises a ground plane, the first and second electrodes being positioned on opposing surfaces of the substrate.

46. The system of claim 3, further comprising a scanner, wherein the scanner comprises:
a transmitter for transmitting the wireless request signal to the sensor;
a receiver for receiving the wireless reply signal from the sensor; and
a processor for decoding the reply signal.

47. The system of claim 46, wherein the scanner further comprises a data storage element for storing the decoded reply.

48. The system of claim 46, wherein the scanner further comprises a display for displaying the decoded reply.

49. The system of claim 46, wherein the scanner further comprises a communications port for exporting the decoded reply.

50. The system of claim 1, wherein the member is a flexible tube for carrying a fluid, and further comprising a second sensing element, wherein the first and second sensing elements are each affixed to one of an inner surface and an outer surface of the flexible tube, each sensing element being positioned at a known distance along a longitudinal axis of the flexible tube.

51. The system of claim 50, wherein the first sensing element produces a measure relating to a change in the radius of the tube, and the second sensing element produces a measure relating to a pressure in the tube.

52. The system of claim 51, wherein the system is configured to measure blood pressure and cardiac stroke volume in a blood vessel.

53. The system of claim 1, wherein the member is a flexible tube for carrying a fluid, and further comprising a second sensor, wherein the first sensor is affixed to one of an inner surface and an outer surface of the flexible tube, and the second sensor overlays the first sensor.

54. The system of claim 1, wherein the member is a flexible tube for carrying at least one of a fluid, gas and solid, the first sensor being affixed to a surface at a closed end of the flexible tube.

55. The system of claim 1, wherein the member is a flexible tube for carrying one of a fluid and a gas, the first sensor being affixed to an inner surface of the tube across a lateral cross section of the tube.

56. A method for measuring distension or bending in a member, the method comprising the steps of:
selecting a sensing element having greater compliance than the member;
conformally affixing the sensing element to a surface of the member; and
measuring an electrical property of the affixed sensing element that varies with distension or bending of the member.

57. The method of claim 56, further comprising the step of electrically coupling the sensing element to a transducer, such that a change in the electrical property of the sensing element causes a change in one of a resonant frequency, amplitude and phase output by the transducer.

58. The method of claim 57, further comprising the step of wirelessly outputting a signal representing said one of a resonant frequency, amplitude and phase output.

59. The method of claim 57, further comprising the steps of:
wirelessly receiving a request signal; and
measuring the electrical property of the sensing element in response to receiving the request signal.

60. The method of claim 59, further comprising the step of validating the request signal before measuring the electrical property.

61. The method of claim 60, wherein the validation step validates the request signal by comparing information in the request signal to a unique identifier.

62. The method of claim 59, further comprising the step of supplying power to the transducer for a predetermined period of time after validating the request signal.

63. The method of claim 58, wherein the output signal contains information relating to a unique indentifier.

64. The method of claim 58, further comprising the steps of:
measuring an electrical property of a reference element;
coupling the reference element to a transducer to produce a resonant frequency output by the transducer; and
wirelessly outputting a signal representing the resonant frequency for the reference element.
